(19) United States
(12) Patent Application Publication
(10) Pub. No.: US 2023/0086718 A1
(43) Pub. Date: Mar. 23, 2023

(54) METHOD FOR PREDICTING THE SURVIVAL TIME OF A PATIENT SUFFERING FROM A CANCER

(71) Applicants: INSERM (INSTITUT NATIONAL DE LA SANTÉ ET DE LA RECHERCHE MÉDICALE), Paris (FR); UNIVERSITÉ DE MONTPELLIER, Montpellier (FR); INSTITUT RÉGIONAL DU CANCER DE MONTPELLIER, Montpellier (FR)

(72) Inventors: Ghita CHABAB, Cedex 5 Montpellier (FR); Virginie LAFONT, Cedex 5 Montpellier (FR); Nathalie BONNEFOY, Cedex 5 Montpellier (FR)

(21) Appl. No.: 17/906,273

(22) PCT Filed: Mar. 19, 2021
(86) PCT No.: PCT/EP2021/057050

§ 371 Date: Sep. 14, 2022

(30) Foreign Application Priority Data
Mar. 20, 2020 (EP) 20305290.7

(51) Int. Cl. G01N 33/574

(52) U.S. Cl. CPC G01N 33/57415; G01N 33/57449; G01N 2333/70596

(57) ABSTRACT

The present invention relates to the prediction of the survival time of a patient suffering from a cancer. The inventors identified in blood from healthy donors a subpopulation of WIT cells that expresses CD73 and displays immunosuppressive phenotype and functions (i.e., production of immunosuppressive molecules and inhibition of αßT cell proliferation). Furthermore, they detected the presence of CD73+ γδ T cells in immune infiltrates of freshly resected breast cancer specimens. Altogether, these data suggest that part of γδ T cells infiltrated in the breast cancer microenvironment presents a regulatory phenotype characterized by CD73 expression, and are likely to display pro-tumor functions through the mechanisms they described in vitro. Thus, the invention relates to a method for predicting the survival time of a patient suffering from a cancer comprising i) determining in a sample obtained from the patient the level of Gamma/Delta T cells expressing CD73 ii) comparing the expression level determined at step i) with its predetermined reference value and iii) providing a good prognosis when the level of Gamma/Delta T cells expressing CD73 determined at step i) is lower than its predetermined reference value, or providing a bad prognosis when the level of Gamma/Delta T cells expressing CD73 determined at step i) is higher than its predetermined reference value.

### FIELD OF THE INVENTION

The present invention relates to a method for predicting the survival time of a patient suffering from a cancer comprising i) determining in a sample obtained from the patient the level of Gamma/Delta T cells expressing CD73 ii) comparing the expression level determined at step i) with its predetermined reference value and iii) providing a good prognosis when the level of Gamma/Delta T cells expressing CD73 determined at step i) is lower than its predetermined reference value, or providing a bad prognosis when the level of Gamma/Delta T cells expressing CD73 determined at step i) is higher than its predetermined reference value.

### BACKGROUND OF THE INVENTION

γδ T cells are non-conventional lymphocytes defined by the expression of a specific receptor composed of the Vγ and Vδ chains. In humans, there are two main subtypes of γδ T cells, according to the expressed Vδ chain: i) Vδ2 T cells, mainly represented by the innate Vγ9Vδ2 T cell subset that is predominant in blood and recognizes phosphorylated non-peptide antigens called phosphoantigens; and ii) non-Vδ2 T cells (mainly represented by the Vδ1 subset) that are predominant in tissues and recognize stress-induced antigens [1-3]. γδ T cells are also found in the tumor environment and are involved in the immune response against many cancers (e.g., myeloma, melanoma, breast, colon, lung, ovary, and prostate) [4-8]. Their anti-tumor effect relies directly on their cytolytic activity against transformed cells and indirectly on their ability to stimulate/regulate the biological functions of other cell types, such as dendritic cells (DC), interferon-γ-producing CD8-positive αβ T cells and natural killer (NK cells) that are required for the initiation and the establishment of an efficient anti-tumor immune response [9-12].

Unlike conventional αβ T cells, γδ T cells: (i) display potent major histocompatibility complex (MHC)-independent reactivity against a broad panel of tumors; (ii) show limited, if any, alloreactivity; and (iii) can be massively and specifically expanded from samples (e.g., peripheral blood). For all these reasons, γδ T cells are considered as highly attractive therapeutic targets for anti-tumor immunotherapies. Although human Vδ1, Vδ2 and Vδ3 T cells display a strong reactivity against tumor cells, γδ T cell-based immunotherapies primarily target the Vδ2 subset due to the facility to expand and activate them with synthetic clinical-grade phosphoantigens (e.g., bromohydrin pyrophosphate) or pharmacological inhibitors (e.g., zoledronate, pamidronate) of the mevalonate pathway that produces these metabolites. Many clinical trials based on the adoptive transfer of ex vivo stimulated Vγ9Vδ2T cells or on their in vivo stimulation using clinical-grade agonists have been carried out in patients with solid cancers or hematological malignancies. The more significant results showed objective responses in 10 to 33% of patients [9-14]. Importantly, many patients who did not respond to treatments exhibited significant and sustained Vγ9Vδ2 T cell proliferation, which raised issues about the presence of suppressive mechanisms that inhibit/divert Vγ9Vδ2 T cell functions and/or their ability to infiltrate tumors.

In line with these observations, the tumor-infiltrating Vγ9Vδ2 T cell abundance, contrary to αβ T cells, is variably associated with the disease outcome [15]. This suggests that mobilization of Vγ9Vδ2 and αβ cells is differentially regulated or that other T cell subsets are involved, such as Vδ1 T cells. Vδ1 T cell population is diverse and through the diversity of its Vδ1 TCRs can recognize the stress-inducible proteins MICA and MICB, which are expressed by some tumor and virus-infected cells [16], glycolipid antigens presented by CD1c [17] and CD1d [18, 19] and the algal protein phycoerythrin [20]. Additionally, Vδ1 T cells can be activated independently of their TCR, via ligation of stimulatory receptors, including NKG2C, NKG2D, NKp30, toll-like receptors, and the β-glucan receptor, dectin 1 [21-25]. Upon activation, Vδ1 T cells proliferate, release cytokines, such as interferon-γ (IFN-γ), tumor necrosis factor-α, and interleukin-17 (IL-17), and chemokines, such as CCL3, CCL4, and CCL5 [21, 22, 26, 27]. Moreover, Vδ1+ T cells are usually predominant (over Vδ2+) in tumor infiltrates, and efficiently react (e.g., cytotoxicity) against tumor cells [5, 28] In spite of their potent anti-tumor properties Vδ1+ T cells had never been tested in the clinic due to lack of suitable expansion/differentiation protocols. Recently, Silva-Santos team developed a new and robust clinical-grade method, for selective and large-scale expansion and differentiation of cytotoxic Vδ1+ T cells and afforded the proof of concept in preclinical models of chronic lymphocytic leukemia [29].

Interestingly, recent in vitro and in vivo data highlighted some degree of γδ T cell polarization driven by environmental signals that can affect their anti-tumor functions. Indeed, some cytokines present in the microenvironment of tumors are known to impact or modify effector functions γδ T cells such as IL-21 [30]. IL-21 is predominantly secreted by natural killer T (NKT) cells, T follicular helper (Tfh) cells and Th17 cells, and plays a role in the differentiation and proliferation of B cells and of CD4+ and CD8+ T lymphocytes [31-33]. IL-21 was also demonstrated to affect Vγ9Vδ2 T cell functions. It has been reported that IL-21 potentiates the cytolytic activity and pro-inflammatory responses of long-term cultured Vγ9Vδ2 T cells [34]. It also favors the differentiation of a Vγ9Vδ2 T cell sub-population into B-helper T cells [35, 36]. On the opposite, we recently demonstrated that IL-21 promote the differentiation of Vγ9Vδ2 T cells with regulatory functions [43] (see below), but the in vivo relevance of such observation remains to be demonstrated.

Besides their anti-tumor functions, γδ T cells have been recently associated with pro-functions in some cancers. Specifically, IL-17-producing γδ T cells have pro-tumoral functions in murine breast, ovarian and hepatocellular cancer models and in human colorectal cancer [37-40]. In human breast cancer, γδ T cell immunosuppressive functions have been associated with DC senescence induction [41]. Moreover, in murine and human pancreatic ductal adenocarcinoma, γδ T cells inhibit αβ T cell activation and infiltration via PDL-1 ligation, thereby allowing tumor progression [42]. Overall, these data support the hypothesis that some γδ T cell subsets can be immunosuppressive and favor tumor progression in selected solid tumor types. In agreement, we recently identified a subset of Vγ9Vδ2 T cells that express the ectonucleotidase CD73 and can produce adenosine, an immunosuppressive molecule. This cell subset also produces IL-10 and IL-8, displays lower effector and cytotoxic functions than CD73-negative Vγ9Vδ2 T cells, and inhibits conventional T cell proliferation in a CD73/adenosine-dependent manner [43].

### SUMMARY OF THE INVENTION

In this study, the inventors extended these observations to Vδ1 T cells. They identified in blood from healthy donors a subpopulation of Vδ1T cells that expresses CD73 (around 20% of whole Vδ1 population) and displays immunosuppressive phenotype and functions (i.e., production of immunosuppressive molecules, such as IL-10, adenosine and the chemotactic factor IL-8, inhibition of αβ T cell proliferation). As shown for Vγ9Vδ2 T cells, the exposure to IL-21, a cytokine found in certain tumor environment, favors the development and amplification of this subset. Then, they performed immunohistochemical (IHC) analysis of a breast tumor tissue microarray (TMA) and observed high γδ T cell infiltration in the tumor microenvironment only of tumors at advanced stages. Furthermore, they detected the presence of CD73+ γδ T cells in immune infiltrates of freshly resected breast cancer specimens. Altogether, these data suggest that part of γδ T cells infiltrated in the breast cancer microenvironment presents a regulatory phenotype characterized by CD73 expression, and are likely to display pro-tumor functions through the mechanisms they described in vitro (i.e., production of adenosine, IL-10 and IL-8), thus favoring an immunosuppressive microenvironment that will promote tumor growth.

Thus, the present invention relates to a method for predicting the survival time of a patient suffering from a cancer comprising i) determining in a sample obtained from the patient the level of Gamma/Delta T cells expressing CD73 ii) comparing the expression level determined at step i) with its predetermined reference value and iii) providing a good prognosis when the level of Gamma/Delta T cells expressing CD73 determined at step i) is lower than its predetermined reference value, or providing a bad prognosis when the level of Gamma/Delta T cells expressing CD73 determined at step i) is higher than its predetermined reference value. Particularly, the invention is defined by its claims.

### DETAILED DESCRIPTION OF THE INVENTION

A first aspect of the invention relates to a method for predicting the survival time of a patient suffering from a cancer comprising i) determining in a sample obtained from the patient the level of Gamma/Delta T cells expressing CD73 ii) comparing the expression level determined at step i) with its predetermined reference value and iii) providing a good prognosis when the level of Gamma/Delta T cells expressing CD73 determined at step i) is lower than its predetermined reference value, or providing a bad prognosis when the level of Gamma/Delta T cells expressing CD73 determined at step i) is higher than its predetermined reference value.

As used herein and according to all aspects of the invention, the term “sample” denotes, blood, peripheral-blood, cancer biopsy or surgical pieces.

In a particular embodiment, the Gamma/Delta T cells expressing CD73 are Gamma/Delta 1 T cells expressing CD73 or Gamma/Delta 2 T cells expressing CD73.

In a particular embodiment, the Gamma/Delta T cells expressing CD73 express also CD39 and/or an immune checkpoint selected in the group consisting in PD-L1, CTLA-4, PD1 or TIGIT.

In a particular embodiment, the Gamma/Delta T cells expressing CD73 express also CD39 and an immune checkpoint selected in the group consisting in PD-L1, CTLA-4, PD1 or TIGIT.

In a particular embodiment, the Gamma/Delta T cells expressing CD73 are Gamma/Delta 2 T cells and express also CD39 and/or an immune checkpoint selected in the group consisting in PD-L1, CTLA-4, PD1 or TIGIT.

In a particular embodiment, the Gamma/Delta T cells expressing CD73 are Gamma/Delta 2 T cells and express also CD39 and an immune checkpoint selected in the group consisting in PD-L1, CTLA-4, PD1 or TIGIT.

In a particular embodiment, the Gamma/Delta T cells expressing CD73 are Gamma/Delta 1 T cells and express also CD39 and PD-L1.

In a particular embodiment, the Gamma/Delta T cells expressing CD73 are Gamma/Delta 2 T cells and express also CD39 and PD-L1.

According to the invention, the term “Gamma/Delta T cells” denotes T cells that have a distinctive T-cell receptor (TCR) on their surface. Most T cells are (alpha beta) T cells with TCR composed of two glycoprotein chains called α (alpha) and β (beta) TCR chains. In contrast, gamma delta (γδ) T cells have a TCR that is made up of one γ (gamma) chain and one δ (delta) chain. This group of T cells is usually less common than T cells in blood, but are at their highest abundance in the gut mucosa, within a population of lymphocytes known as intraepithelial lymphocytes (IELs).

According to the invention, the term “Gamma/Delta 1 T cells” or the term “Gamma/Delta 2 T cells” denotes two sub-types of gamma delta (γδ) T cells with specific δ (delta) chain.

According to the invention, the term “Gamma/Delta T cells expressing CD73 or CD39 or an immune checkpoint” denotes gamma delta (γδ) T cells which express at their surfaces at least one the protein selected in the list consisting in CD73, CD39 or an immune checkpoint. According to the invention, the Gamma/Delta T cells can express at their surfaces CD73 and CD39 or CD73 and an immune checkpoint or CD39 and an immune checkpoint or CD73, CD39 and an immune checkpoint.

As used herein the term “CD39” also known as “ectonucleoside triphosphate diphosphohydrolase-1 or NTPDase1” denotes a typical cell surface enzyme with a catalytic site on the extracellular face. NTPDase1 is an ectonucleotidase that catalyses the hydrolysis of γ- and β-phosphate residues of triphospho- and diphosphonucleosides to the monophosphonucleoside derivative. For example, NTPDase1 hydrolyzes P2 receptor ligands, namely ATP, ADP, UTP and UDP with similar efficacy.

As used herein the term “CD73” also known as “ecto-5′-nucleotidase” denotes an enzyme that in humans is encoded by the NTSE gene. CD73 commonly serves to convert AMP to adenosine.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to adrenal cortical cancer, anal cancer, bile duct cancer, bladder cancer, bone cancer, brain and central nervous system cancer, breast cancer, Castleman disease, cervical cancer, colorectal cancer, endometrial cancer, oesophagus cancer, gallbladder cancer, gastrointestinal carcinoid tumors, Hodgkin's disease, non-Hodgkin's lymphoma, lymphoma, leukemia, myeloma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

In some embodiment, the cancer is breast cancer, ovarian cancer, pancreatic cancer or colorectal cancer.

As used herein, the term “patient” denotes a human with a cancer according to the invention.

As used herein, the term “survival time” denotes the percentage of people in a study or treatment group who are still alive for a certain period of time after they were diagnosed with or started treatment for a disease, such as a cancer (according to the invention). The survival time rate is often stated as a five-year survival rate, which is the percentage of people in a study or treatment group who are alive five years after their diagnosis or the start of treatment.

As used herein and according to the invention, the term “survival time” can regroup the term OS.

As used herein, the term “Overall survival (OS)” denotes the time from diagnosis of a disease such as a cancer (according to the invention) until death from any cause. The overall survival rate is often stated as a two-year survival rate, which is the percentage of people in a study or treatment group who are alive two years after their diagnosis or the start of treatment.

In one embodiment, the invention relates to a method for predicting the survival time of a patient suffering from a cancer comprising i) determining in a sample obtained from the patient the level of Gamma/Delta T cells expressing CD73 and CD39 ii) comparing the expression level determined at step i) with its predetermined reference value and iii) providing a good prognosis when the level of Gamma/Delta T cells expressing CD73 and CD39 determined at step i) is lower than its predetermined reference value, or providing a bad prognosis when the level of Gamma/Delta T cells expressing CD73 and CD39 determined at step i) is higher than its predetermined reference value.

In another embodiment, the invention relates to a method for predicting the survival time of a patient suffering from a cancer comprising i) determining in a sample obtained from the patient the level of Gamma/Delta T cells expressing CD73 and an immune checkpoint selected in the group consisting in PD-L1, CTLA-4, PD1 or TIGIT ii) comparing the expression level determined at step i) with its predetermined reference value and iii) providing a good prognosis when the level of Gamma/Delta T cells expressing CD73 and the immune checkpoint determined at step i) is lower than its predetermined reference value, or providing a bad prognosis when the level of Gamma/Delta T cells expressing CD73 and the immune checkpoint determined at step i) is higher than its predetermined reference value.

In another embodiment, the invention relates to a method for predicting the survival time of a patient suffering from a cancer comprising i) determining in a sample obtained from the patient the level of Gamma/Delta T cells expressing CD73, CD39 and an immune checkpoint selected in the group consisting in PD-L1, CTLA-4, PD1 or TIGIT ii) comparing the expression level determined at step i) with its predetermined reference value and iii) providing a good prognosis when the level of Gamma/Delta T cells expressing CD73, CD39 and the immune checkpoint determined at step i) is lower than its predetermined reference value, or providing a bad prognosis when the level of Gamma/Delta T cells expressing CD73, CD39 and the immune checkpoint determined at step i) is higher than its predetermined reference value.

Measuring the level of Gamma/Delta T cells expressing the markers of the invention can be done by measuring the gene expression level of the markers or by measuring the protein expression level of the markers and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlshad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat anti-avidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. .1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a non-fluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

It will be appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 nm) and a second specific binding agent (in this case an anti-DNP antibody, or antibody fragment, labelled with a second fluorophore (for example, a second spectrally distinct QUANTUM DOT®, e.g., that emits at 705 nm). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR (or q RT-PCR).

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a patient sample, to another sample, or between samples from different sources.

Measuring the level of Gamma/Delta T cells expressing the markers of the invention can be done by measuring the protein expression level of the markers (protein level) and can be performed by a variety of techniques well known in the art.

According to the invention, the level of the marker may be measured at the surface of the Gamma/Delta T cells cells in blood or tissues for example.

Typically protein concentration (or marker level as used in the invention) may be measured for example by capillary electrophoresis-mass spectroscopy technique (CE-MS) or ELISA performed on the sample.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS). etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Methods of the invention may comprise a step consisting of comparing the proteins and fragments concentration in circulating cells with a control value. As used herein, “concentration of protein” refers to an amount or a concentration of a transcription product. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the markers of the protein.

In a particular embodiment, the level of the Gamma/Delta T cells can be performed by flow cytometry. When this method is used, the method consists of determining the amount of the markers of the invention (CD73 and/or CD39 and/or an immune checkpoint according to the invention) expressed on the Gamma/Delta T cells. According to the invention and the flow cytometry method, when the fluorescence intensity is high or bright, the level of the markers on the Gamma/Delta T cells is high and thus the level of Gamma/Delta T cells is high and when the fluorescence intensity is low or dull, the level of the markers on the Gamma/Delta T cells is low and thus the level of Gamma/Delta T cells is low.

Others methods like mass cytometry, Immunohistochemistry (IHC), in situ hybridization (ISH) and imagery by mass cytometry can be used to determine the level of the Gamma/Delta T.

According to the invention, the term “level of the Gamma/Delta T cells” denotes also the percentage of Gamma/Delta T cells among other cells or the number (quantity) of Gamma/Delta T cells.

Predetermined reference values used for comparison of the level of Gamma/Delta T cells may comprise “cut-off” or “threshold” values that may be determined as described herein. Each reference (“cut-off”) value for Gamma/Delta T cells level may be predetermined by carrying out a method comprising the steps of

a) providing a collection of samples from patients suffering of a cancer;

b) determining the level of the f Gamma/Delta T cells according to the invention for each sample contained in the collection provided at step a);

c) ranking the tumor tissue samples according to said level

d) classifying said samples in pairs of subsets of increasing, respectively decreasing, number of members ranked according to their expression level,

e) providing, for each sample provided at step a), information relating to the actual clinical outcome for the corresponding cancer patient;

f) for each pair of subsets of samples, obtaining a Kaplan Meier percentage of survival curve;

g) for each pair of subsets of samples calculating the statistical significance (p value) between both subsets

h) selecting as reference value for the level, the value of level for which the p value is the smallest.

For example the level of Gamma/Delta T cells has been assessed for 100 cancer samples of 100 patients. The 100 samples are ranked according to their expression level. Sample 1 has the best expression level and sample 100 has the worst expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate cancer samples and therefore the corresponding patients.

Kaplan-Meier curves of percentage of survival as a function of time are commonly used to measure the fraction of patients living for a certain amount of time after treatment and are well known by the man skilled in the art.

The man skilled in the art also understands that the same technique of assessment of the expression level of a protein should of course be used for obtaining the reference value and thereafter for assessment of the expression level of a protein of a patient subjected to the method of the invention.

Such predetermined reference values of expression level may be determined for any Gamma/Delta T cells or markers defined above.

A further object of the invention relates to kits for performing the methods of the invention, wherein said kits comprise means for measuring the level of Gamma/Delta T cells in the sample obtained from the patient.

The kits may include antibodies, probes, primers macroarrays or microarrays as above described. For example, the kit may comprise a set of probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

The present invention also relates to the Gamma/Delta T cells as biomarker for outcome of cancer patients.

Thus, in another aspect, the invention also relates to a method for treating a cancer in a patient with a bad prognosis as described above comprising the administration to said patient of an anti-cancer treatment.

Anti-cancer treatment can use an anti-cancer agents. Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In another embodiment, the anti-cancer treatment can be radiotherapy or brachytherapy.

In yet another embodiment, the anti-cancer agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA-4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA-4 antibodies, anti-PD1 antibodies, anti-PD-L1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1. Analysis of γδ T cell populations in fresh human breast cancer samples. (A) Summary dot plot (mean±SEM of 16 tumor samples) showing the percentage of alive CD45+ cells in tumors, CD3+ lymphocytes among the CD45+ cells, γδ T cells among the CD3+ cells, CD39+ γδ T cells among all γδ T cells and CD73+ γδ T cells among all γδ T cells. (B) Summary dot plot (mean±SEM of 12 tumor samples) showing the percentages of Vδ1 and Vδ2 cells (in the total γδ T population), of CD39+ cells and CD73+ cells in each subpopulation.

FIG. 2: Expression of CD39 and CD73 in expanded Vol T cells. Summary dot plot showing the percentage of CD39+ (A), CD73+ (B) and CD39+/CD73+ cells (C) in control (CTL; only rhIL-2) and Vδ1 T cells expanded in the presence of rhIL-2 and rhIL-21 (IL-21). Cumulative data are from 10 (A and B) and 7 (C) healthy individuals; ns, not significant, *p<0.05; **p<0.005 (paired Wilcoxon test).

FIG. 3. Expression of PD-L1 in expanded Vδ1 T cells. Summary dot plot showing the percentage of PD-L1+ cells in total Vδ1 T cells and in the CD73+ Vδ1 T cells expanded in the presence of rhIL-2 and rhIL-21 (IL-21) or not (CTL; only rhIL-2). ns, not significant, **p<0.005 (paired Wilcoxon test).

FIG. 4. Analysis of γδ T cell populations in fresh human breast cancer samples. (A) Summary dot plot (mean±SEM of 16 tumor samples) showing the percentage of CD45+ cells among living cells, CD3+ lymphocytes among the CD45+ cells, γδ T cells among the CD3+ cells. (B) Summary dot plot (mean±SEM of 16 tumor samples) showing the percentage of CD73+, CD39+ and PD-L1 among all γδ T cells and CD39+ and PD-L1+ cells among CD73+ γδ T cells

FIG. 5. Expression of IL-8 and IL-10 in tumor-infiltrating γδ T cells. Cumulative data of 8 breast cancer patients showing the percentage of IL-8 positive cells and the MFI in CD73- and CD73+ γδ T cells (upper panel). Cumulative data of 7 breast cancer patients showing the percentage of IL-10 positive cells and the MFI in CD73- and CD73+ γδ T cells (lower panel), *p<0.05; **p<0.005 (paired Wilcoxon test).

FIG. 6. Tumor-infiltrating γδ and CD73+ γδ T lymphocyte densities in ovarian cancer patients are increased in short-term surviving patients. Cumulative data of 91 ovarian cancer patients with 43 Short-term survivors (ST) and 49 Long-term survivors (LT) showing the number of γδ T cells (A) and CD73+ γδ T cells (B), ***p<0.001; ****p<0.0001 (Mann-Whitney t test).

FIG. 7. Tumor-infiltrating γδ and CD73+ γδ T cells predict worse clinical outcome in ovarian cancer patients. The Kaplan-Meier survival curves/log-rank tests were used to compare OS in groups with high and low numbers of γδ T cells (A) and with high and low number of CD73+ γδ T cells (B).

### EXAMPLES

### Example 1

Material & Methods

Antigens, Antibodies and Reagents

Phytohemagglutinin (PHA) was purchased from Thermofisher Scientific. The anti-human CD3 (UCHT1), and the anti-human TCR γδ and TCR Vδ1 antibodies were purchased from Beckman Coulter (Brea, Calif., USA). Anti-human CD3, CD73, CD39 and their isotypically matched control mouse antibodies were from BD Biosciences (San Jose, Calif., USA). The anti-human TCR γδ antibody used for IHC was from Santa Cruz Biotechnology (USA). Recombinant human IL-2 (rhIL-2) was from Novartis Pharma (Rueil-Malmaison, France), and recombinant IL-21 (rhIL-21) was from Miltenyi Biotec (Paris, France). ELISA kits for detecting human IL-10 and IL-8 were from BD Biosciences (San Jose, Calif., USA). ELISA kit for the detection of human TGFβ was from RD system.

Cell Culture

Peripheral Blood Mononuclear Cells (PBMCs) were obtained by density centrifugation on Ficoll-Paque (Eurobio, Les Ulis, France) of blood samples from healthy donors and breast cancer patients. Healthy donor samples were provided by the Etablissement Français du Sang (Convention EFS-OCPM no 21PLER2018-0069) and the blood of patients was provided by the ICM (BCB-EC-1-FR-ICM-ENR-269-004-CRB). To analyze comparable cohorts between cancer patients and healthy donors, we selected EFS blood samples from healthy women with an age ranging from 18 to 70 years. Vδ1 T cells were isolated from PBMCs by a positive immunoselection using the anti-human Vδ1 antibody (Beckman Coulter) and anti-IgG1 magnetic beads (Miltenyi Biotec). Briefly, 300.106 cells were incubated with 10 μg of anti-human Vδ1 antibody in 5 ml of PBS supplemented with 2% SVF and EDTA (2 mM) for 1 hour at 4° C., then washed and incubated with 200 μl of anti-IgG1 magnetic beads for 1 hour at 4° C., then washed and collected on column according to the manufacturer's instructions (Miltenyi Biotec). Reproducible high purity of Vδ1 T cells (>90%) was obtained with this protocol. Purified Vδ1 T cells (2.106 cells/ml) were stimulated with 2 μg/mL of PHA in the presence of syngeneic macrophages isolated using their adherence properties. Briefly, PBMC (2.106/ml) were incubated in RPMI 10% FCS for 1 hour at 37° C. in 96 well-plates to allow to monocytes to adhere and differentiate in macrophages. Non-adherent cells were removed by 2 washes with RPMI medium. Purified Vδ1 T cells were added to macrophages, activated by PHA and expanded in the complete medium containing RPMI 1640/Glutamax medium supplemented with 5% human AB serum and 5% fetal calf serum (FCS) in the presence of rhIL-2 (control) or rhIL-2+rhIL-21 (experimental condition) at 37° C. in humidified atmosphere with 5% CO2. Every 2 days, fresh medium is added and after 1 week Vδ1 T cells were separated from adherent macrophages and amplified in the complete medium with cytokines for 2 more weeks before phenotyping and analysis.

Flow Cytometry Analysis

Cells were first incubated at 4° C. for 30 min with Fc-block solution to minimize non-specific binding of antibodies to Fc receptors, then incubated with a dye cell viability and the panel of antibodies in the staining buffer (PBS-2% FCS) at 4° C. for 30 min, cells were then washed and fixed in 1% paraformaldehyde. Data were acquired with a Cytoflex cytometer (Beckman-Coulter) and analyzed with the FlowJo software.

ELISA

Expanded Vδ1 T cells (2.106 cells/mL) were incubated in fresh medium without cytokines in wells coated or not with the anti-CD3 antibody (UCHT1) for 6 h, and then supernatants were collected. IL-8 and IL-10 protein levels were assessed using the relevant BD Biosciences Opteia Kits. The mean values of duplicate samples from the same experiment are shown for each data point with their standard error of the mean (SEM).

Adenosine Measurement by MALDI TOF Spectrometry

Amplified Vδ1 T cells (2.106 cells/mL) were washed in cold PBS and resuspended in PBS supplemented with 50 mM AMP (Sigma) at 4° C. for 30 min. After centrifugation, adenosine levels in supernatants were analyzed by MALDI-TOF mass spectrometry, as previously described by Bastid et al. [44].

T Cell Proliferation Assay

Peripheral blood from healthy donors was obtained from the EFS and mononuclear cells were isolated on a Ficoll gradient. PBMC were stained with 2.5 μM CFSE for 11 min at 37° C. 4×104 CFSE stained PBMC were distributed in 96 well flat-bottom plates coated with the anti-CD3 antibody (UCHT1 10 μg/ml). Sorted CD73- and CD73+ Vδ1 T cells were added at ratio 1:1. Proliferation of αβ T cells was analyzed by flow cytometry at day 5.

Tumor Dissociation

Freshly resected tumors from patients were cut into small pieces (around 1 mm3). Tissues were resuspended in digestion solution (10 mg/ml collagenase IV from Sigma and 10 mg/ml DNase I from Roche) in Hanks modified balanced salt solution and alternate between enzymatic digestion (15 min at 37° C.) and mechanical dissociation using the gentle MACS dissociator (Miltenyi Biotec) for 3 rounds. The obtained single cell suspensions were washed in PBS/2% FCS, and resuspended in PBS/2% FCS with FcBlock (Miltenyi Biotec) at 4° C. in the dark for 30 min. Then, cells were washed and incubated with a panel of conjugated antibodies and results analyzed as described in the previous sections. The cession of fresh samples was approved by the Montpellier Cancer Institute Review Board (ICM-CORT-2018-34).

Breast Tumor Tissue Microarray

A TMA that included breast tumors from 50 patients was constructed using two malignant tissue cores (1 mm diameter) per tumor. Tissue samples were from patients who underwent surgery at our institution between 2001 and 2011 and received no neoadjuvant treatment. They were informed and gave their consent for using their tissue samples for biological research. Tumor samples were collected following the French laws under the supervision of an investigator and declared to the French Ministry of Higher Education and Research (declaration number DC-2008-695). The study was approved by the Montpellier Cancer Institute Review Board (ICM-CORT-2015-32).

Immunohistochemistry

After epitope retrieval in EDTA buffer (pH 9) and neutralization of endogenous peroxidase, 3 μm TMA sections were incubated with the mouse monoclonal anti-TCR γδ antibody (Clone H-41, Santa Cruz) at room temperature for 30 min. This was followed by an amplification step with a mouse linker and the standard detection system (Flex, Dako), consisting of a dextran backbone to which a large number of peroxidase molecules and secondary anti-mouse and anti-rabbit antibodies were coupled. 3,30-Diaminobenzidine was used as substrate. The NanoZoomer slide scanner system (Hamamatsu Photonics) was used to digitalize the stained TMA sections with a ×20 objective. Immunoreactive cells were manually identified and counted on the digitalized slides with the NDP.view software. When both samples from the same tumor were assessable (39 out of 50), the mean value was calculated and data expressed as number of TCR γδ-positive cells per spot. Salgado's method was used to determine the immune infiltrate in TMA spots [45].

Statistical Analysis

Results were compared using the paired Wilcoxon test or a chi2 test depending on the experiment. A P value <0.05 was considered as statistically significant. Analyses were performed using the GraphPad Prism software, version 6.

Results

A Subset of Vδ1 T Cells Expresses the CD39 and CD73 Ectonucleotidases

Extracellular ATP and adenosine act as positive and negative regulators of the immune response, respectively. We previously reported that ectonucleoside triphosphate diphosphohydrolase 1 (ENTPD1 or CD39), which catalyzes the phosphohydrolysis of extracellular ATP into ADP and of ADP into AMP, is expressed by Vγ9Vδ2 T cells after TCR activation [43]. Conversely, the ecto-5′-nucleotidase CD73, which completes AMP conversion into adenosine, is only expressed by a subset of activated Vγ9Vδ2 T cells, a phenotype that can be amplified in the presence of IL-21 [43]. Here, we investigated CD39 and CD73 expression in the Vδ1 T cell subpopulation. To this aim, we isolated Vδ1 T cells from the blood of healthy donors, and activated and amplified them in vitro in the presence of rhIL-2 (control) or rhIL-2+rhIL21 (experimental condition). We observed that most Vδ1 T cells (>85%) expressed CD39. Moreover, exposure to rhIL-21 did not modify the percentage of CD39-positive cells, suggesting that IL-21 does not influence its expression (data not shown). Conversely, rhIL-21 significantly increased the fraction of CD73-positive Vδ1 T cells to 50% (vs 20% in the control condition), although results were heterogeneous among donors (data not shown). The fraction of CD39/CD73-positive Vδ1 T cells also was significantly higher in cells exposed to rhIL-21 compared with control cells (40% vs 18%) (data not shown).

Adenosine Production by Vδ1 T Cells

Around 60-70% of CD73-positive Vδ1 T cells expressed also CD39 (data not shown), suggesting that these cells could produce adenosine in an autonomous manner in an ATP-rich environment. As the percentage of CD39-positive Vδ1 T cells was identical in the presence/absence of rhIL-21, we hypothesized that CD73 expression was the only parameter influencing adenosine production. Therefore, we quantified adenosine levels in the supernatants of Vδ1 T cells by MALDI-TOF mass spectrometry, as previously described [44]. IL-21-amplified Vδ1 T cells generated higher amounts of adenosine in the presence of AMP compared with control cells (only rhIL-2) (˜1.4 μM vs ˜0.4 μM) (data not shown). The presence of adenosine 5′-(α,β-methylene)-diphosphate sodium salt (APCP), a pharmacological inhibitor of CD73, strongly impaired adenosine production by control and IL-21-amplified Vδ1 T cells (data not shown), confirming that adenosine synthesis by these cells depends on CD73 activity. Taken together, these results suggest that CD73-positive Vδ1 T cells display an immunosuppressive function through adenosine production. Moreover, the presence of some CD73-positive control cells suggests that rhIL-21 promotes the development of a preexisting immunosuppressive population, rather than just inducing CD73 expression.

CD73+ Vδ1 T Cells Produce IL-10 and IL-8

Conventional regulatory T cells inhibit the immune response by producing adenosine and also by secreting immunoregulatory cytokines, such as IL-10 and TGF-β [46]. Therefore, we determined whether Vδ1 T cells produce regulatory cytokines. In absence of activation we detected basal level of IL-10 in the supernatants of IL-21-amplified Vδ1 T cells whether they expressed or not CD73, but not of control cells. TCR/CD3 activation strongly increased IL-10 production by IL-21-amplified CD73+ but not in the CD73− Vδ1 T cells. (data not shown). Conversely, we did not detect TGF-β secretion in any of the tested conditions (data not shown).

As many studies showed that immunosuppressive T cells produce CXCL8 or IL-8, a chemokine implicated in angiogenesis and the recruitment of neutrophils and MDSCs in the tumor microenvironment, we investigated IL-8 production by Vδ1 T cells [40, 47, 48]. As observed for IL-10, IL-8 was highly present only in activated IL-21-amplified CD73+ Vδ1 T cells (data not shown). Thus, CD73+ Vδ1 T cell subset amplified in the presence of IL-21 were the main producer of IL-10 and IL-8.

These results suggest that through the secretion of IL-10 and IL-8, Vδ1 T cells could contribute to create a tumor microenvironment rich in suppressive factors that favor tumor development and growth.

CD73+ Vδ1 T Cells Inhibit αβ T Cell Proliferation

To determine whether CD73+ Vδ1 T cells display regulatory functions, we analyzed their capacity to inhibit γδ T cells proliferation. We sorted Vδ1 T cells in function to their CD73 expression and then evaluated proliferation of CFSE-labeled T cells grown in the presence or not of immobilized anti-CD3 monoclonal antibodies and co-cultured with CD73− or CD73+ Vδ1 T cells. Around 90% of αβ T cell proliferated when co-cultured with CD73− Vδ1 T cells compared to only 60% in the presence of CD73+ subset (data not shown). Overall, these results suggested that CD73+ Vδ1 T cells display regulatory functions that can affect the proliferative capacity of other immune cells, such as αβ T cells.

Identification of γδ T cells in human breast cancer samples

To evaluate the in vivo relevance of our findings in human solid cancers, we first assessed by IHC γδ TCR expression in a TMA that included 50 human breast samples at different SBR grades and phenotypes based on HER2 and hormone receptor expression status. All tumor samples used to build TMA were collected at surgery from patients who did not receive any neoadjuvant treatment. Among the 50 tumors analyzed after IHC, 41 showed at least one γδT lymphocyte per mm2. Although γδ T lymphocyte density was highly heterogeneous among samples (from 1 to >500 γδ T cells per mm2) their density progressively increased from grade I to grade III (data not shown). This suggests, as previously reported by others [49], that in breast cancer, the presence of γδ T cells is associated with late tumor grade and/or poor prognosis. Nevertheless, in parallel the analysis of immune infiltrate by Salgado's method showed that lower γδ TCR expression are associated with lower immune infiltrates and inversely. Moreover, although the difference did not reach significance, we observed a higher frequency of γδ T cells in triple-negative breast cancer (TNBC) compared to HER2- or hormone receptor breast cancer (data not shown). TNBCs comprise a very heterogeneous group of cancers. The general prognosis is rather similar with other breast cancer of same stage, except that more aggressive treatment is required due to the inefficacy of hormone- or HER2-targeting therapies. Some types of triple-negative breast cancer are known to be more aggressive, with poor prognosis, while other types have very similar or better prognosis than hormone receptor positive breast cancers. This suggests that other parameters must intervene in the prognostic and evolution of breast cancer.

Presence of CD73+ γδ T Cells in Human Breast Cancer

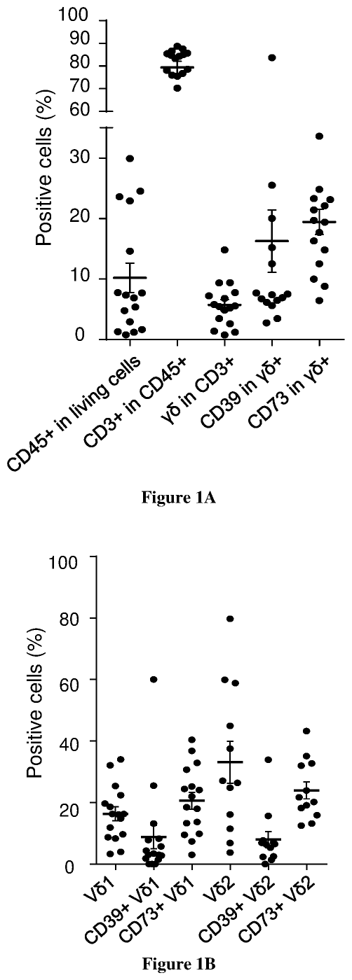

Then, to characterize the phenotype of infiltrated γδ T cells in human breast tumors, we dissociated fresh breast cancer biopsies and analyzed the phenotype of tumor-infiltrating γδ T cells by flow cytometry with the gating protocol. Tumor samples (n=16) were from patients with grade I, II and III breast tumor who did not receive any neoadjuvant treatment. Although the fraction of CD45-positive cells in breast cancers was very heterogeneous (0.75% to 25%), the proportion of CD3-positive cells among the CD45-positive cells was homogenous (around ˜80%), and γδ T cells were always present and represented 3% to 13% of CD3+ cells. (FIG. 1A). Moreover, around 17 and 20% of γδ T cells expressed CD39 and CD73 respectively (FIG. 1A), suggesting that γδ T cells with potential suppressive functions are present in breast cancer. To better characterize the γδ T cell phenotype, we analyzed the presence of the Vδ1 and Vδ2 subsets within the total γδ T cell population in 12 of these 16 tumors. Overall, the percentage of Vδ2 cells tended to be higher than that of Vδ1 T cells (not significant), whereas the percentage of CD73+ cells (about 20%) was similar in both populations (FIG. 1B).

### Example 2

Material & Methods

Cell Culture

Peripheral Blood Mononuclear Cells (PBMCs) were obtained by density centrifugation on Ficoll-Paque (Eurobio, Les Ulis, France) of blood samples from healthy donors and breast cancer patients. Healthy donor samples were provided by the Etablissement Français du Sang and the blood of patients was provided by the Institut regional du Cancer de Montpellier (ICM). To analyze comparable cohorts between cancer patients and healthy donors, we selected EFS blood samples from healthy women with an age ranging from 18 to 70 years. Vδ1 T cells were isolated from PBMCs by a positive immunoselection using the anti-human Vδ1 antibody (Beckman Coulter) and anti-IgG1 magnetic beads (Miltenyi Biotec). Briefly, 300.106 cells were incubated with 10 μg of anti-human Vδ1 antibody in 5 ml of PBS supplemented with 2% SVF and EDTA (2 mM) for 1 hour at 4° C., then washed and incubated with 200 μl of anti-IgG1 magnetic beads for 1 hour at 4° C., then washed and collected on column according to the manufacturer's instructions (Miltenyi Biotec). Reproducible high purity of Vδ1 T cells (>90%) was obtained with this protocol. Purified Vδ1 T cells (2.106 cells/ml) were stimulated with 2 μg/mL of PHA in the presence of syngeneic macrophages isolated using their adherence properties. Briefly, PBMC (2.106/ml) were incubated in RPMI 10% FCS for 1 hour at 37° C. in 96 well-plates to allow to monocytes to adhere and differentiate in macrophages. Non-adherent cells were removed by 2 washes with RPMI medium. Purified Vδ1 T cells were added to macrophages, activated by PHA and expanded in the complete medium containing RPMI 1640/Glutamax medium supplemented with 5% human AB serum and 5% fetal calf serum (FCS) in the presence of rhIL-2 (control) or rhIL-2+rhIL-21 (experimental condition) at 37° C. in humidified atmosphere with 5% CO2. Every 2 days, fresh medium is added and after 1 week Vδ1 T cells were separated from adherent macrophages and amplified in the complete medium with cytokines for 2 more weeks before phenotyping and analysis.

Flow Cytometry Analysis

Cells were first incubated at 4° C. for 30 min with Fc-block solution to minimize non-specific binding of antibodies to Fc receptors, then incubated with a dye cell viability and the panel of antibodies in the staining buffer (PBS-2% FCS) at 4° C. for 30 min, cells were then washed and fixed in 1% paraformaldehyde. Data were acquired with a Cytoflex cytometer (Beckman-Coulter) and analyzed with the FlowJo software.

Tumor Dissociation

Freshly resected tumors from patients were cut into small pieces (around 1 mm3). Tissues were resuspended in digestion solution (10 mg/ml collagenase IV from Sigma and 10 mg/ml DNase I from Roche) in Hanks modified balanced salt solution and alternate between enzymatic digestion (15 min at 37° C.) and mechanical dissociation using the gentle MACS dissociator (Miltenyi Biotec) for 3 rounds. The obtained single cell suspensions were washed in PBS/2% FCS, and resuspended in PBS/2% FCS with FcBlock (Miltenyi Biotec) at 4° C. in the dark for 30 min. Then, cells were washed and incubated with a panel of conjugated antibodies and results analyzed as described in the previous sections. The cession of fresh samples was approved by the Montpellier Cancer Institute Review Board (ICM-CORT-2018-34).

Results

A Subset of Vol T Cells Expresses the CD39, CD73 and PD-L1

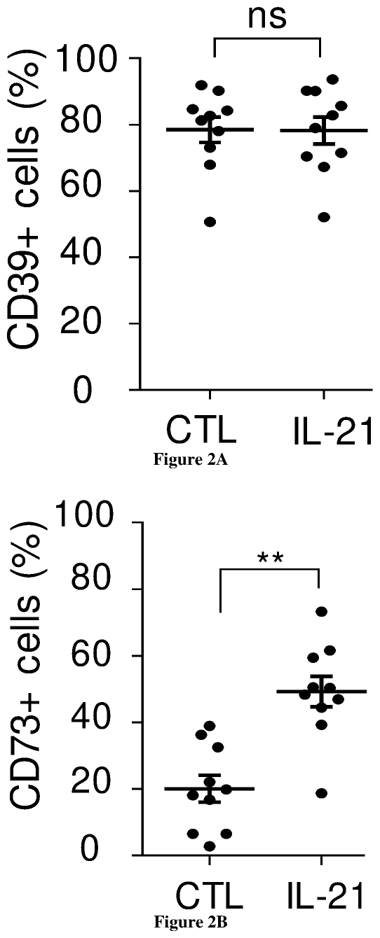

We investigated CD39, CD73 and PD-L1 expression in the Vδ1 T cell subpopulation. To this aim, we isolated Vδ1 T cells from the blood of healthy donors, and activated and amplified them in vitro in the presence of rhIL-2 (control) or rhIL-2+rhIL21 (experimental condition). We observed that most Vδ1 T cells (>85%) expressed CD39. Moreover, exposure to rhIL-21 did not modify the percentage of CD39-positive cells, suggesting that IL-21 does not influence its expression (FIG. 2A). Conversely, rhIL-21 significantly increased the fraction of CD73-positive Vδ1 T cells to 50% (vs 20% in the control condition), although results were heterogeneous among donors (FIG. 2B). The fraction of CD39/CD73-positive Vδ1 T cells also was significantly higher in cells exposed to rhIL-21 compared with control cells (40% vs 18%) (FIG. 2C).

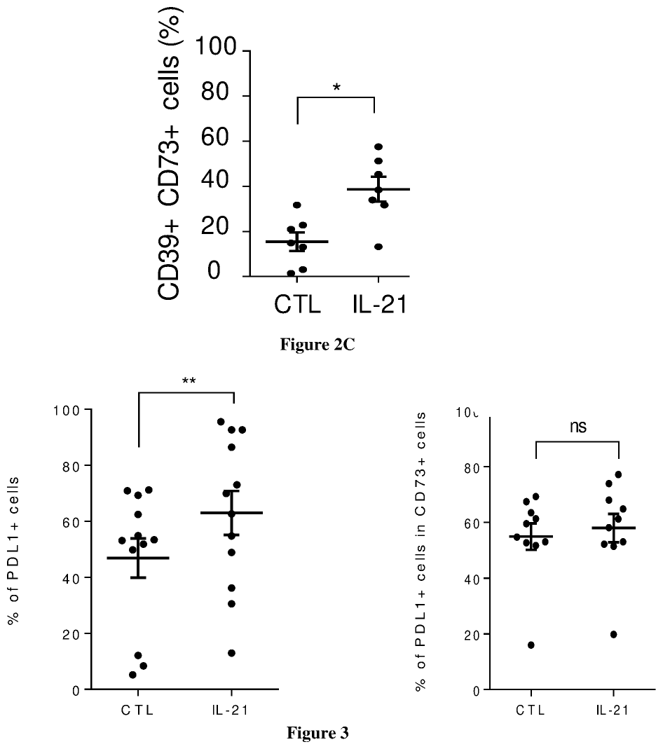

Also, we observed that about 50% of Vol T cells (in the control condition) expressed PD-L1 (FIG. 3) and this fraction of Vδ1 T cells increased in cells exposed to rhIL-21 (65%). The percentage of PD-L1+ in CD73-positive Vδ1 T cells was around 60% and the exposure of rhIL-21 did not modify it (FIG. 3).

Presence of Various CD73+ γδ T Cell Subsets in Human Breast Cancer

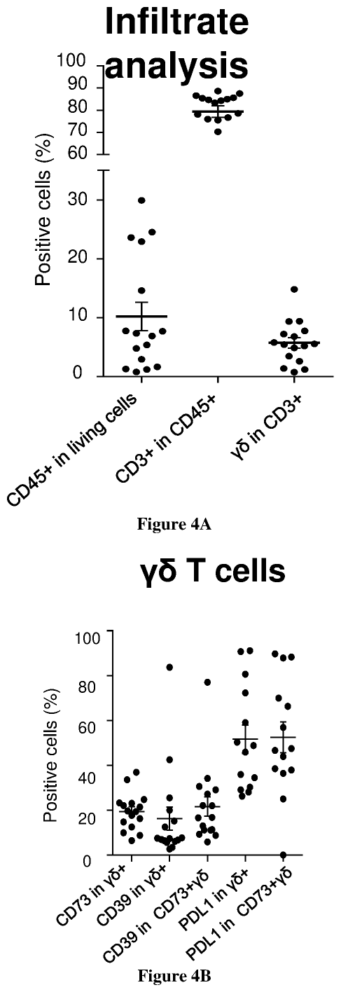

Then, to characterize the phenotype of infiltrated γδ T cells in human breast tumors, we dissociated fresh breast cancer biopsies and analyzed the phenotype of tumor-infiltrating γδ T cells by flow cytometry with the gating protocol. Tumor samples (n=16) were from patients with grade I, II and III breast tumor who did not receive any neoadjuvant treatment. Although the fraction of CD45-positive cells in breast cancers was very heterogeneous (0.75% to 25%), the proportion of CD3-positive cells among the CD45-positive cells was homogenous (around ˜80%), and γδ T cells were always present and represented 3% to 13% of CD3+ cells. (FIG. 4A). Moreover, around 17 and 20% of γδ T cells expressed CD39 and CD73 respectively (FIG. 4B) and 50% of γδ T cells and CD73+ γδ T cells expressed PD-L1, suggesting that γδ T cells with potential suppressive functions are present in breast cancer.

To conclude, we demonstrated in human breast tumors the presence of various CD73+ γδ T cell subsets expressing immunosuppressive markers such as CD39 and CD73 ectonucleotidases and inhibitory immunocheckpoint PD-L1 (data not shown). These various CD73+ γδ T cell subsets could be used as a prognostic marker of the tumor evolution (data not shown).

### Example 3

Material & Methods

Tumor Dissociation

Freshly resected tumors from patients were cut into small pieces (around 1 mm3). Tissues were resuspended in digestion solution (10 mg/ml collagenase IV from Sigma and 10 mg/ml DNase I from Roche) in Hanks modified balanced salt solution and alternate between enzymatic digestion (15 min at 37° C.) and mechanical dissociation using the gentle MACS dissociator (Miltenyi Biotec) for 3 rounds. The obtained single cell suspensions were washed in PBS/2% FCS, resuspended at 10·10⁶ cells/ml in RPM/10% FCS and incubated in the presence of Golgi STOP (Bd Biosciences) at 37° C. for 4 hours.

The cession of fresh samples was approved by the Montpellier Cancer Institute Review Board (ICM-CORT-2018-34).

Flow Cytometry Analysis

Cells were harvested and incubated at 4° C. for 30 min with Fc-block solution to minimize non-specific binding of antibodies to Fc receptors. After washing cells were incubated with a dye cell viability and the panel of antibodies (anti-CD45, -CD3, -γδTCR, -CD73) for extracellular staining in the staining buffer (PBS-2% FCS) at 4° C. for 30 min, before fixation and permeabilization with the BD FixPerm Kit and intracellular staining for IL-10 and IL-8. Data were acquired on a Cytoflex cytometer (Beckman Coulter) and results analyzed using the FlowJo software.

Results

Expression of the immunosuppressive cytokines IL-10 and IL-8 in CD73- versus CD73+ tumor-infiltrating γδ T lymphocytes (γδ TILs).

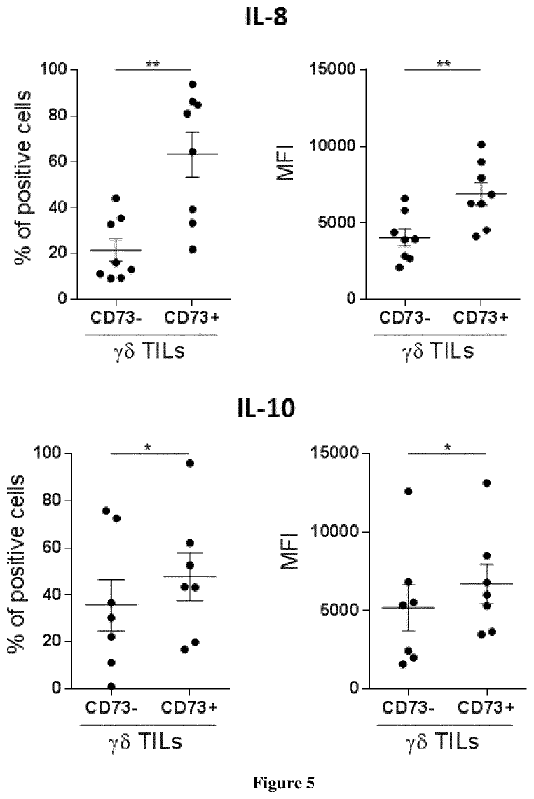

To analyze the functional suppressive activity of CD73+ γδ TILs, we investigated, ex vivo by flow cytometry, the expression of IL-10 and IL-8 in infiltrated γδ T cells in human breast tumors obtained from patients who did not receive any neoadjuvant treatment (n=8 for IL-8 analyses and n=7 for IL-10 analyses). We assessed the expression of IL-10 and IL-8 in CD73− and CD73+ γδ subsets. Around 20% and 30% of CD73− γδ T cells expressed IL-8 and IL-10 respectively (FIG. 5). Percentages of IL-8 and IL-10 positive cells were significantly increased in the CD73+ γδ T cell sub-population. Furthermore, MFI of IL-8 and IL-10 positive cells were significantly increased in the CD73+ γδ T cells compared to the CD73-subpopulation. Altogether these results demonstrate the increased suppressive capacity of CD73+ γδ T cell subset in breast cancer patients.

### Example 4

Material and Methods.

Sample Collection

Tissue samples were selected from the biological resource center of Montpellier Cancer Institute (ICM). Clinical data were obtained by reviewing the medical files. Samples were collected following the French laws under the supervision of an investigator and their collection was declared to the French Ministry of Higher Education and Research. The study was approved by the ICM Institutional Review Board (ICM-CORT-2020-32).

Ovarian Cancer Tissue Microarray

Two TMA with a total of 91 ovarian cancer samples were constructed for retrospective studies allowing the comparison of long-term vs short-term ovarian cancer survivors. For each tumor sample, two cores (1 mm in diameter) were sampled from different malignant areas.

Breast Cancer Tissue Microarray

A tissue microarray (TMA) with breast tumor samples from 50 chemotherapy-naive patients was constructed using two malignant tissue cores (1 mm diameter) per tumor.

Immunofluorescence

After de-paraffinization, TMA sections were subjected to antigen retrieval using 1× Target Retrieval Solution (Dako, S2367), then incubated in 1× Superblock Blocking Buffer (Thermofisher, 37515) for 45 min followed by 1 h incubation in a FcBlock solution (Miltenyi Biotech—130-059-901). After washing, TMA sections were incubated with primary antibodies against TCR γδ (H-41, Santa Cruz, 1/25) and CD73 (D7AF9A, CST, 1/100) overnight at 4° C. After washing, sections were incubated with secondary antibodies: goat anti-Rabbit Alexa Fluor Plus 555 (Thermofisher, A32732) and goat anti-Mouse Alexa Fluor 647 (Thermofisher, A21236). Finally, sections were counterstained with DAPI and were imaged with AxioScan (Zeiss) to obtain high-power field images. TMA sections were analyzed by observer blinded to the clinicopathological characteristics and patient outcomes at the time of scoring.

Statistical Analysis Data are presented as scatter plots showing the mean values with the standard error of the mean (SEM). Results were compared using Mann-Whitney t test. A P value <0.05 was considered statistically significant. Analyses were performed using GraphPad Prism, version 6. *** p<0.001; ****p<0.0001

For survival analysis, categorical variables were presented as frequency distributions and continuous variables as medians and ranges. Categorical variables were compared with the Pearson's chi-square test. OS was defined as the time between the date of surgery and the date of death (whatever the cause). Patients lost to follow-up were censored at the last documented visit. The Kaplan-Meier method was used to estimate the OS. Differences in survival rates were compared using the log-rank test. Multivariate analyses were performed using the Cox proportional hazard model. Hazard ratios (HR) are given with their 95% confidence interval (95% CI). Statistical analyses were performed with STATA 16.0 (StatCorp, College Station, Tex., USA).

Results

Total γδ and CD73+ γδ Tumor Infiltrating Lymphocyte (TIL) Density are Increased in Short-Term Survival Ovarian Cancer Patients

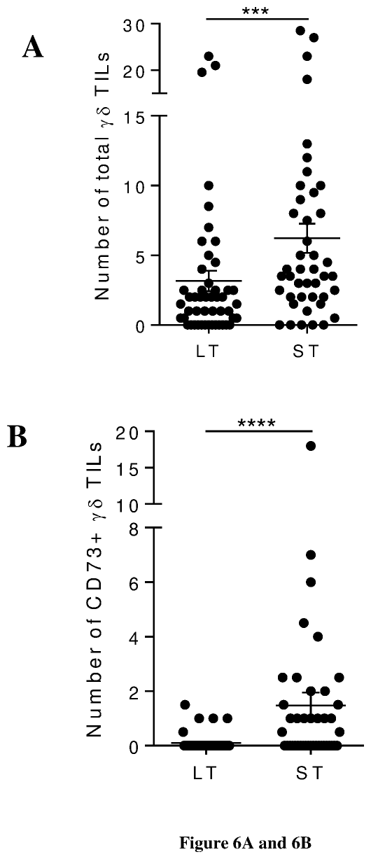

We quantified by immunofluorescence both total γδ TILs and CD73+ γδ TILs in tumor samples from 91 ovarian cancer patients. Patients were classified in two groups: long-term (LT) and short-term (ST) survivors. We observed significantly higher densities of total γδ TILs and CD73+ γδ TILs in ST patients than in LT patients suggesting that γδ and CD73+ γδ T cell presence at the tumor site are associated with poor-prognosis in ovarian cancer patients (FIGS. 6A and 6B).

CD73+ γδ TILs Predict Worse Clinical Outcome in Ovarian Cancer

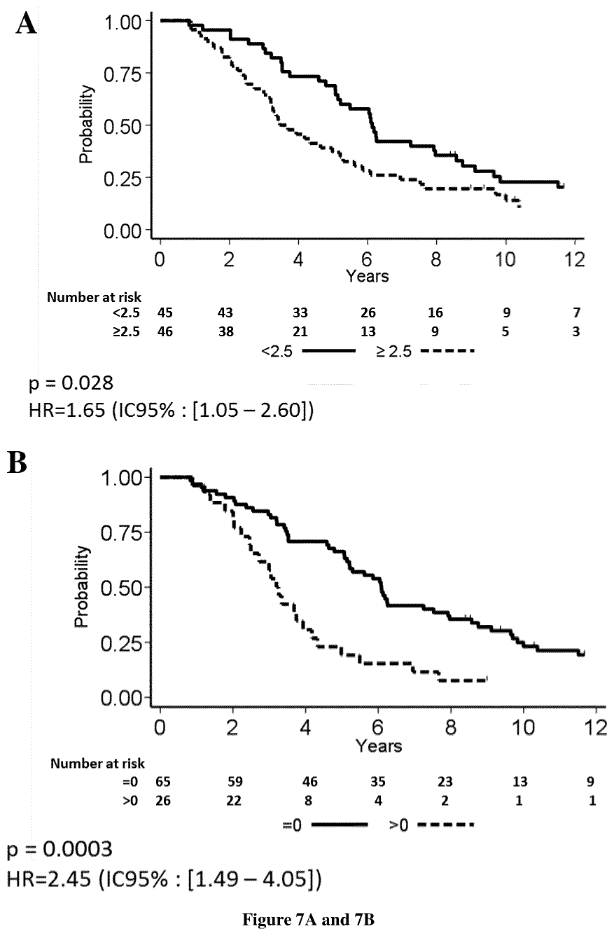

Because tumor-infiltrating CD73+ γδ T cells were predominant in ST surviving patients, we next investigated the association between these cells and clinical outcome of the patients, focusing on the overall survival (OS). The median follow-up was 13.44 years (95% CI [11.67-15.47]) and the median survival was 5.14 years (95% CI [3.75-6.08]). We analyzed total γ6 TILs, CD73+ γδ TILs and clinical data from 91 patients. Patients with a low density of γδ T cells at the tumor site had significantly longer OS compared with those with a high density of γδ T cells (p=0.028) (FIG. 7A). This result is even more pronounced when looking at CD73+γδ TIL subset. Patients with high density of CD73+ γδ T cells had significantly shorter OS compared with patients with a low density of CD73+ γδ T cells (p=0.0003) (FIG. 7B). Thus, the presence of CD73+ γδ TILs could be used a prognosis factor in ovarian cancer.

Tumor-Infiltrating CD73+ γδ T Predict Worse Clinical Outcome in Breast Cancer

We investigate the association between the presence of CD73+ γδ TILs and the clinical outcome of breast cancer patients. We analyze CD73+ γδ TILs and clinical data from 50 patients at different SBR grades and phenotypes based on HER2 and hormone receptor expression status. Patients with high density of CD73+ γδ T cells have a shorter OS compared with those with a low density of CD73+ γδ T cells. These results on breast cancer patients are corroborated by the study realized by Hu et al. [65] where they also showed that the presence of tumor infiltrating CD73+γδ T cells is a bad prognosis for breast cancer patients. These results reinforce the idea that the CD73+ γδ T cell presence in breast tumor is a strong prognosis factor.

### REFERENCES

Throughout this application, various references describe the state of the art to which this invention pertains. The disclosures of these references are hereby incorporated by reference into the present disclosure.
1. Adams, E. J., Gu, S., Luoma, A. M. (2015) Human gamma delta T cells: Evolution and ligand recognition. Cellular immunology 296, 31-40.
2. Kabelitz, D. (2011) gammadelta T-cells: cross-talk between innate and adaptive immunity. Cellular and molecular life sciences: CMLS 68, 2331-3.
3. Vantourout, P. and Hayday, A. (2013) Six-of-the-best: unique contributions of gammadelta T cells to immunology. Nature reviews. Immunology 13, 88-100.
4. Bouet-Toussaint, F., Cabillic, F., Toutirais, O., Le Gallo, M., Thomas de la Pintiere, C., Daniel, P., Genetet, N., Meunier, B., Dupont-Bierre, E., Boudjema, K., Catros, V. (2008) Vgamma9Vdelta2 T cell-mediated recognition of human solid tumors. Potential for immunotherapy of hepatocellular and colorectal carcinomas. Cancer immunology, immunotherapy: CII 57, 531-9.
5. Cordova, A., Toia, F., La Mendola, C., Orlando, V., Meraviglia, S., Rinaldi, G., Todaro, M., Cicero, G., Zichichi, L., Donni, P. L., Caccamo, N., Stassi, G., Dieli, F., Moschella, F. (2012) Characterization of human gammadelta T lymphocytes infiltrating primary malignant melanomas. PloS one 7, e49878.
6. Corvaisier, M., Moreau-Aubry, A., Diez, E., Bennouna, J., Mosnier, J. F., Scotet, E., Bonneville, M., Jotereau, F. (2005) V gamma 9V delta 2 T cell response to colon carcinoma cells. Journal of immunology 175, 5481-8.
7. Meraviglia, S., Eberl, M., Vermijlen, D., Todaro, M., Buccheri, S., Cicero, G., La Mendola, C., Guggino, G., D'Asaro, M., Orlando, V., Scarpa, F., Roberts, A., Caccamo, N., Stassi, G., Dieli, F., Hayday, A. C. (2010) In vivo manipulation of Vgamma9Vdelta2 T cells with zoledronate and low-dose interleukin-2 for immunotherapy of advanced breast cancer patients. Clinical and experimental immunology 161, 290-7.
8. Raspollini, M. R., Castiglione, F., Rossi Degl'innocenti, D., Amunni, G., Villanucci, A., Garbini, F., Baroni, G., Taddei, G. L. (2005) Tumour-infiltrating gamma/delta T-lymphocytes are correlated with a brief disease-free interval in advanced ovarian serous carcinoma. Annals of oncology: official journal of the European Society for Medical Oncology 16, 590-6.
9. Lo Presti, E., Pizzolato, G., Gulotta, E., Cocorullo, G., Gulotta, G., Dieli, F., Meraviglia, S. (2017) Current Advances in gammadelta T Cell-Based Tumor Immunotherapy. Frontiers in immunology 8, 1401.
10. Marquez-Medina, D., Salla-Fortuny, J., Salud-Salvia, A. (2012) Role of gamma-delta T-cells in cancer: another opening door to immunotherapy. Clinical & translational oncology: official publication of the Federation of Spanish Oncology Societies and of the National Cancer Institute of Mexico 14, 891-5.
11. Pauza, C. D., Liou, M. L., Lahusen, T., Xiao, L., Lapidus, R. G., Cairo, C., Li, H. (2018) Gamma Delta T Cell Therapy for Cancer: It Is Good to be Local. Frontiers in immunology 9, 1305.
12. Pressey, J. G., Adams, J., Harkins, L., Kelly, D., You, Z., Lamb, L. S., Jr. (2016) In vivo expansion and activation of gammadelta T cells as immunotherapy for refractory neuroblastoma: A phase 1 study. Medicine 95, e4909.
13. Buccheri, S., Guggino, G., Caccamo, N., Li Donni, P., Dieli, F. (2014) Efficacy and safety of gammadeltaT cell-based tumor immunotherapy: a meta-analysis. Journal of biological regulators and homeostatic agents 28, 81-90.
14. Gomes, A. Q., Martins, D. S., Silva-Santos, B. (2010) Targeting gammadelta T lymphocytes for cancer immunotherapy: from novel mechanistic insight to clinical application.

Cancer research 70, 10024-7.
15. Tosolini, M., Pont, F., Poupot, M., Vergez, F., Nicolau-Travers, M. L., Vermijlen, D., Sarry, J. E., Dieli, F., Fournie, J. J. (2017) Assessment of tumor-infiltrating TCRVgamma9Vdelta2 gammadelta lymphocyte abundance by deconvolution of human cancers microarrays. Oncoimmunology 6, e1284723.
16. Groh, V., Steinle, A., Bauer, S., Spies, T. (1998) Recognition of stress-induced MHC molecules by intestinal epithelial gammadelta T cells. Science 279, 1737-40.
17. Spada, F. M., Grant, E. P., Peters, P. J., Sugita, M., Melian, A., Leslie, D. S., Lee, H. K., van Donselaar, E., Hanson, D. A., Krensky, A. M., Majdic, O., Porcelli, S. A., Morita, C. T., Brenner, M. B. (2000) Self-recognition of CD1 by gamma/delta T cells: implications for innate immunity. The Journal of experimental medicine 191, 937-48.
18. Bai, L., Picard, D., Anderson, B., Chaudhary, V., Luoma, A., Jabri, B., Adams, E. J., Savage, P. B., Bendelac, A. (2012) The majority of CD1d-sulfatide-specific T cells in human blood use a semiinvariant Vdelta1 TCR. European journal of immunology 42, 2505-10.
19. Uldrich, A. P., Le Nours, J., Pellicci, D. G., Gherardin, N. A., McPherson, K. G., Lim, R. T., Patel, O., Beddoe, T., Gras, S., Rossjohn, J., Godfrey, D. I. (2013) CD1d-lipid antigen recognition by the gammadelta TCR. Nature immunology 14, 1137-45.
20. Zeng, X., Wei, Y. L., Huang, J., Newell, E. W., Yu, H., Kidd, B. A., Kuhns, M. S., Waters, R. W., Davis, M. M., Weaver, C. T., Chien, Y. H. (2012) gammadelta T cells recognize a microbial encoded B cell antigen to initiate a rapid antigen-specific interleukin-17 response. Immunity 37, 524-34.
21. Fausther-Bovendo, H., Wauquier, N., Cherfils-Vicini, J., Cremer, I., Debre, P., Vieillard, V. (2008) NKG2C is a major triggering receptor involved in the V[delta]1 T cell-mediated cytotoxicity against HIV-infected CD4 T cells. Aids 22, 217-26.
22. Hudspeth, K., Fogli, M., Correia, D. V., Mikulak, J., Roberto, A., Della Bella, S., Silva-Santos, B., Mavilio, D. (2012) Engagement of NKp30 on Vdelta1 T cells induces the production of CCL3, CCL4, and CCL5 and suppresses HIV-1 replication. Blood 119, 4013-6.
23. Kuroda, H., Saito, H., Ikeguchi, M. (2012) Decreased number and reduced NKG2D expression of Vdelta1 gammadelta T cells are involved in the impaired function of Vdelta1 gammadelta T cells in the tissue of gastric cancer. Gastric cancer: official journal of the International Gastric Cancer Association and the Japanese Gastric Cancer Association 15, 433-9.
24. Maher, C. O., Dunne, K., Comerford, R., O'Dea, S., Loy, A., Woo, J., Rogers, T. R., Mulcahy, F., Dunne, P. J., Doherty, D. G. (2015) Candida albicans stimulates IL-23 release by human dendritic cells and downstream IL-17 secretion by Vdelta1 T cells. Journal of immunology 194, 5953-60.
25. Wesch, D., Peters, C., Oberg, H. H., Pietschmann, K., Kabelitz, D. (2011) Modulation of gammadelta T cell responses by TLR ligands. Cellular and molecular life sciences: CMLS 68, 2357-70.
26. Boullier, S., Dadaglio, G., Lafeuillade, A., Debord, T., Gougeon, M. L. (1997) V delta 1 T cells expanded in the blood throughout HIV infection display a cytotoxic activity and are primed for TNF-alpha and IFN-gamma production but are not selected in lymph nodes. Journal of immunology 159, 3629-37.
27. Fenoglio, D., Poggi, A., Catellani, S., Battaglia, F., Ferrera, A., Setti, M., Murdaca, G., Zocchi, M. R. (2009) Vdelta1 T lymphocytes producing IFN-gamma and IL-17 are expanded in HIV-1-infected patients and respond to Candida albicans. Blood 113, 6611-8.
28. Lo Presti, E., Dieli, F., Meraviglia, S. (2014) Tumor-Infiltrating gammadelta T Lymphocytes: Pathogenic Role, Clinical Significance, and Differential Programing in the Tumor Microenvironment. Frontiers in immunology 5, 607.
29. Almeida, A. R., Correia, D. V., Fernandes-Platzgummer, A., da Silva, C. L., da Silva, M. G., Anjos, D. R., Silva-Santos, B. (2016) Delta One T Cells for Immunotherapy of Chronic Lymphocytic Leukemia: Clinical-Grade Expansion/Differentiation and Preclinical Proof of Concept. Clinical cancer research: an official journal of the American Association for Cancer Research 22, 5795-5804.
30. Lindner, S., Dahlke, K., Sontheimer, K., Hagn, M., Kaltenmeier, C., Barth, T. F., Beyer, T., Reister, F., Fabricius, D., Lotfi, R., Lunov, O., Nienhaus, G. U., Simmet, T., Kreienberg, R., Moller, P., Schrezenmeier, H., Jahrsdorfer, B. (2013) Interleukin 21-induced granzyme B-expressing B cells infiltrate tumors and regulate T cells. Cancer research 73, 2468-79.
31. Leonard, W. J. and Wan, C. K. (2016) IL-21 Signaling in Immunity. F1000Research 5.
32. Spolski, R. and Leonard, W. J. (2008) Interleukin-21: basic biology and implications for cancer and autoimmunity. Annual review of immunology 26, 57-79.
33. Croce, M., Rigo, V., Ferrini, S. (2015) IL-21: a pleiotropic cytokine with potential applications in oncology. Journal of immunology research 2015, 696578.
34. Thedrez, A., Harly, C., Morice, A., Salot, S., Bonneville, M., Scotet, E. (2009) IL-21-mediated potentiation of antitumor cytolytic and proinflammatory responses of human V gamma 9V delta 2 T cells for adoptive immunotherapy. J Immunol 182, 3423-31.
35. Eberl, M., Engel, R., Beck, E., Jomaa, H. (2002) Differentiation of human gamma-delta T cells towards distinct memory phenotypes. Cellular immunology 218, 1-6.
36. Caccamo, N., Todaro, M., La Manna, M. P., Sireci, G., Stassi, G., Dieli, F.

(2012) IL-21 regulates the differentiation of a human gammadelta T cell subset equipped with B cell helper activity. PLoS One 7, e41940.
37. Coffelt, S. B., Kersten, K., Doornebal, C. W., Weiden, J., Vrijland, K., Hau, C. S., Verstegen, N. J., Ciampricotti, M., Hawinkels, L. J., Jonkers, J., de Visser, K. E. (2015) IL-17-producing gammadelta T cells and neutrophils conspire to promote breast cancer metastasis. Nature 522, 345-8.
38. Ma, S., Cheng, Q., Cai, Y., Gong, H., Wu, Y., Yu, X., Shi, L., Wu, D., Dong, C., Liu, H. (2014) IL-17A produced by gammadelta T cells promotes tumor growth in hepatocellular carcinoma. Cancer research 74, 1969-82.
39. Rei, M., Goncalves-Sousa, N., Lanca, T., Thompson, R. G., Mensurado, S., Balkwill, F. R., Kulbe, H., Pennington, D. J., Silva-Santos, B. (2014) Murine CD27(−) Vgamma6(+) gammadelta T cells producing IL-17A promote ovarian cancer growth via mobilization of protumor small peritoneal macrophages. Proceedings of the National Academy of Sciences of the United States of America 111, E3562-70.
40. Wu, P., Wu, D., Ni, C., Ye, J., Chen, W., Hu, G., Wang, Z., Wang, C., Zhang, Z., Xia, W., Chen, Z., Wang, K., Zhang, T., Xu, J., Han, Y., Zhang, T., Wu, X., Wang, J., Gong, W., Zheng, S., Qiu, F., Yan, J., Huang, J. (2014) gammadeltaT17 cells promote the accumulation and expansion of myeloid-derived suppressor cells in human colorectal cancer. Immunity 40, 785-800.
41. Peng, G., Wang, H. Y., Peng, W., Kiniwa, Y., Seo, K. H., Wang, R. F. (2007) Tumor-infiltrating gammadelta T cells suppress T and dendritic cell function via mechanisms controlled by a unique toll-like receptor signaling pathway. Immunity 27, 334-48.
42. Daley, D., Zambirinis, C. P., Seifert, L., Akkad, N., Mohan, N., Werba, G., Barilla, R., Torres-Hernandez, A., Hundeyin, M., Mani, V. R. K., Avanzi, A., Tippens, D., Narayanan, R., Jang, J. E., Newman, E., Pillarisetty, V. G., Dustin, M. L., Bar-Sagi, D., Hajdu, C., Miller, G. (2016) gammadelta T Cells Support Pancreatic Oncogenesis by Restraining alphabeta T Cell Activation. Cell 166, 1485-1499 e15.
43. Barjon, C., Michaud, H. A., Fages, A., Dejou, C., Zampieri, A., They, L., Gennetier, A., Sanchez, F., Gros, L., Eliaou, J. F., Bonnefoy, N., Lafont, V. (2017) IL-21 promotes the development of a CD73-positive Vgamma9Vdelta2 T cell regulatory population. Oncoimmunology 7, e1379642.
44. Bastid, J., Regairaz, A., Bonnefoy, N., Dejou, C., Giustiniani, J., Laheurte, C., Cochaud, S., Laprevotte, E., Funck-Brentano, E., Hemon, P., Gros, L., Bec, N., Larroque, C., Alberici, G., Bensussan, A., Eliaou, J. F. (2015) Inhibition of CD39 enzymatic function at the surface of tumor cells alleviates their immunosuppressive activity. Cancer immunology research 3, 254-65.
45. Salgado, R., Denkert, C., Demaria, S., Sirtaine, N., Klauschen, F., Pruneri, G., Wienert, S., Van den Eynden, G., Baehner, F. L., Penault-Llorca, F., Perez, E. A., Thompson, E. A., Symmans, W. F., Richardson, A. L., Brock, J., Criscitiello, C., Bailey, H., Ignatiadis, M., Floris, G., Sparano, J., Kos, Z., Nielsen, T., Rimm, D. L., Allison, K. H., Reis-Filho, J. S., Loibl, S., Sotiriou, C., Viale, G., Badve, S., Adams, S., Willard-Gallo, K., Loi, S., International, T. W. G. (2015) The evaluation of tumor-infiltrating lymphocytes (TILs) in breast cancer: recommendations by an International TILs Working Group 2014. Annals of oncology: official journal of the European Society for Medical Oncology 26, 259-71.
46. Yano, H., Andrews, L. P., Workman, C. J., Vignali, D. A. A. (2019) Intratumoral regulatory T cells: markers, subsets and their impact on anti-tumor immunity. Immunology 157, 232-247.
47. Waugh, D. J. and Wilson, C. (2008) The interleukin-8 pathway in cancer. Clinical cancer research: an official journal of the American Association for Cancer Research 14, 6735-41.
48. Xie, K. (2001) Interleukin-8 and human cancer biology. Cytokine & growth factor reviews 12, 375-91.
49. Ma, C., Zhang, Q., Ye, J., Wang, F., Zhang, Y., Wevers, E., Schwartz, T., Hunborg, P., Varvares, M. A., Hoft, D. F., Hsueh, E. C., Peng, G. (2012) Tumor-infiltrating gammadelta T lymphocytes predict clinical outcome in human breast cancer. Journal of immunology 189, 5029-36.
50. Elliott, M. R., Chekeni, F. B., Trampont, P. C., Lazarowski, E. R., Kadl, A., Walk, S. F., Park, D., Woodson, R. I., Ostankovich, M., Sharma, P., Lysiak, J. J., Harden, T. K., Leitinger, N., Ravichandran, K. S. (2009) Nucleotides released by apoptotic cells act as a find-me signal to promote phagocytic clearance. Nature 461, 282-6.
51. Aymeric, L., Apetoh, L., Ghiringhelli, F., Tesniere, A., Martins, I., Kroemer, G., Smyth, M. J., Zitvogel, L. (2010) Tumor cell death and ATP release prime dendritic cells and efficient anticancer immunity. Cancer research 70, 855-8.
52. Silva-Vilches, C., Ring, S., Mahnke, K. (2018) ATP and Its Metabolite Adenosine as Regulators of Dendritic Cell Activity. Frontiers in immunology 9, 2581.
53. White, N. and Burnstock, G. (2006) P2 receptors and cancer. Trends in pharmacological sciences 27, 211-7.
54. Novitskiy, S. V., Ryzhov, S., Zaynagetdinov, R., Goldstein, A. E., Huang, Y., Tikhomirov, O. Y., Blackburn, M. R., Biaggioni, I., Carbone, D. P., Feoktistov, I., Dikov, M. M. (2008) Adenosine receptors in regulation of dendritic cell differentiation and function. Blood 112, 1822-31.
55. Zarek, P. E., Huang, C. T., Lutz, E. R., Kowalski, J., Horton, M. R., Linden, J., Drake, C. G., Powell, J. D. (2008) A2A receptor signaling promotes peripheral tolerance by inducing T-cell anergy and the generation of adaptive regulatory T cells. Blood 111, 251-9.
56. Cekic, C., Day, Y. J., Sag, D., Linden, J. (2014) Myeloid expression of adenosine A2A receptor suppresses T and NK cell responses in the solid tumor microenvironment. Cancer research 74, 7250-9.
57. Ohta, A., Kini, R., Ohta, A., Subramanian, M., Madasu, M., Sitkovsky, M. (2012) The development and immunosuppressive functions of CD4(+) CD25(+) FoxP3(+) regulatory T cells are under influence of the adenosine-A2A adenosine receptor pathway. Frontiers in immunology 3, 190.
58. de Andrade Mello, P., Coutinho-Silva, R., Savio, L. E. B. (2017) Multifaceted Effects of Extracellular Adenosine Triphosphate and Adenosine in the Tumor-Host Interaction and Therapeutic Perspectives. Frontiers in immunology 8, 1526.
59. Mannino, M. H., Zhu, Z., Xiao, H., Bai, Q., Wakefield, M. R., Fang, Y. (2015) The paradoxical role of IL-10 in immunity and cancer. Cancer letters 367, 103-7.
60. Emmerich, J., Mumm, J. B., Chan, I. H., LaFace, D., Truong, H., McClanahan, T., Gorman, D. M., Oft, M. (2012) IL-10 directly activates and expands tumor-resident CD8(+) T cells without de novo infiltration from secondary lymphoid organs. Cancer research 72, 3570-81.
61. Oft, M. (2014) IL-10: master switch from tumor-promoting inflammation to antitumor immunity. Cancer immunology research 2, 194-9.
62. Wu, Y., Kyle-Cezar, F., Woolf, R. T., Naceur-Lombardelli, C., Owen, J., Biswas, D., Lorenc, A., Vantourout, P., Gazinska, P., Grigoriadis, A., Tutt, A., Hayday, A. (2019) An innate-like Vdelta1(+) gammadelta T cell compartment in the human breast is associated with remission in triple-negative breast cancer. Science translational medicine 11.
63. Pizzolato, G., Kaminski, H., Tosolini, M., Franchini, D. M., Pont, F., Martins, F., Valle, C., Labourdette, D., Cadot, S., Quillet-Mary, A., Poupot, M., Laurent, C., Ysebaert, L., Meraviglia, S., Dieli, F., Merville, P., Milpied, P., Dechanet-Merville, J., Fournie, J. J. (2019) Single-cell RNA sequencing unveils the shared and the distinct cytotoxic hallmarks of human TCRVdelta1 and TCRVdelta2 gammadelta T lymphocytes. Proceedings of the National Academy of Sciences of the United States of America 116, 11906-11915.
64. Bono, M. R., Fernandez, D., Flores-Santibanez, F., Rosemblatt, M., Sauma, D. (2015) CD73 and CD39 ectonucleotidases in T cell differentiation: Beyond immunosuppression. FEBS letters 589, 3454-60.
65. Hu, G., Cheng, P., Pan, J., Wang, S., Ding, Q., Jiang, Z., Cheng, L., Shao, X., Huang, L., Huang, J. (2020) An IL6-Adenosine Positive Feedback Loop between CD73+ γδTregs and CAFs Promotes Tumor Progression in Human Breast Cancer. Cancer Immunol Res. (10):1273-1286.

1. A method for predicting the survival time of and treating a patient suffering from a cancer comprising
i) measuring the level of Gamma/Delta T cells expressing CD73 in a sample obtained from the patient;
ii) determining that the level of Gamma/Delta T cells expressing CD73 is higher than a reference value and
iii) treating the subject determined to have a higher level of Gamma/Delta T cells expressing CD73.

2. The method according to claim 1 wherein the Gamma/Delta 1 T cells expressing CD73 are Gamma/Delta 1 T cells expressing CD73 or Gamma/Delta 2 T cells expressing CD73.

3. The method according to claim 1, wherein the Gamma/Delta T cells expressing CD73 also express CD39 and/or an immune checkpoint selected from the group consisting of PD-L1, CTLA-4, PD1 and TIGIT.

4. The method according to claim 3 wherein the Gamma/Delta T cells expressing CD73 also express CD39 and an immune checkpoint selected from the group consisting of PD-L1, CTLA4, PD1 and TIGIT.

5. The method according to claim 4 wherein the Gamma/Delta T cells expressing CD73 are Gamma/Delta 1 T cells and also express CD39 and PD-L1.

6. The method according to claim 4 wherein the Gamma/Delta T cells expressing CD73 are Gamma/Delta 2 T cells and also express CD39 and PD-L1.

7. The method according to claim 1, wherein the sample is blood, peripheral-blood, a cancer biopsy or surgical pieces.

8. The method according to claim 1, wherein the step of measuring the Gamma/Delta T cells is done by flow cytometry.

9. (canceled)
